(19) United States
(12) Patent Application Publication
(10) Pub. No.: US 2025/0146732 A1
(43) Pub. Date: May 8, 2025

(54) REFRIGERATION APPLIANCE AND METHOD OF MAKING SAME

(71) Applicant: BSH Hausgeräte GmbH, München (DE)

(72) Inventors: Tong ZHOU, Chuzhou (CN); Xiupeng CHEN, Nanjing (CN); Yanjie ZHANG, Nanjing (CN)

(21) Appl. No.: 18/891,732

(22) Filed: Sep. 20, 2024

(30) Foreign Application Priority Data
Nov. 7, 2023 (CN) 202311477267.5

(51) Int. Cl. F25D 17/04; F25D 17/08

(52) U.S. Cl. CPC F25D 17/042; F25D 17/08; F25D 2317/041

(57) ABSTRACT

A refrigeration appliance has: a compartment, provided with a storage space applicable to accommodating articles to be cooled; an air duct module, applicable to forming an air duct in the compartment, and including an air duct main body applicable to defining the air duct, and an air duct cover installed to one side of the air duct main body facing the storage space; and a purification module, at least partially located in the air duct to purify gas flowing through the air duct. The air duct module is configured to allow the purification module to be installed to the air duct cover from one side of the air duct main body facing away from the air duct cover in a state where the air duct main body and the air duct cover are assembled with each other.

### CROSS-REFERENCE TO RELATED APPLICATION

This application claims the priority, under 35 U.S.C. § 119, of Chinese Patent Application CN 202311477267.5, filed Nov. 7, 2023; the prior application is herewith incorporated by reference in its entirety.

### FIELD AND BACKGROUND OF THE INVENTION

This application relates to the field of household appliances, and in particular to a refrigeration appliance and a method of making the same.

Modules applicable to improving gas quality are disposed in refrigeration appliances. Taking a refrigerator as an example, a purification module can be disposed in an air duct of the refrigerator. To install the purification module in the air duct, complex operations are usually required.

The purification module is usually installed to an air duct cover plate, and at least a portion of the purification module needs to be located in the air duct so as to purify gas in the air duct. In an existing refrigerator, usually, a purification module is first installed to an air duct cover plate, and then one or more components applicable to forming an air duct are assembled to the air duct cover plate and the purification module. This assembly process is complicated and has high requirements for an operator. Otherwise, the one or more components are prone to damage in the assembly process. Conversely, when the purification module is disassembled, it is necessary to first disassemble the one or more components applicable to forming the air duct. The disassembly process of the purification module is also very complicated.

### SUMMARY OF THE INVENTION

An objective of embodiments of this application is to provide a refrigeration appliance and a method of making the same to overcome at least one of the above-mentioned shortcomings of the prior art.

According to a first aspect of this application, an embodiment of this application provides a refrigeration appliance, provided with: a compartment, provided with storage space applicable to accommodating articles to be cooled; an air duct module, applicable to forming an air duct in the compartment, and including an air duct main body applicable to defining the air duct, and an air duct cover installed to one side of the air duct main body facing the storage space; and a purification module, at least partially located in the air duct to purify gas flowing through the air duct. The air duct module is configured to allow the purification module to be installed to the air duct cover from one side of the air duct main body facing away from the air duct cover in a state where the air duct main body and the air duct cover are assembled with each other.

Thus, the assembly process and/or disassembly process of the air duct module and the purification module can be simplified. When the refrigeration appliance is assembled, the air duct module can be provided, for example, by a manufacturer of the air duct module as an assembled whole. An assembly worker of the refrigeration appliance can directly operate an assembled air duct module, without performing assembly operations of the air duct main body and the air duct cover. Thus, difficulty of assembly operations can be reduced. Similarly, in the disassembly process, difficulty of the operation can be also reduced by way of the present invention. In addition, the air duct module can be prevented from being damaged in the assembly process and/or disassembly process.

According to an optional embodiment of this application, the purification module can be configured to be installed to the air duct cover from the side of the air duct main body facing away from the air duct cover in a direction perpendicular to a main plane of the air duct cover. Thus, the purification module can be simpler to install, without complicating a structure of the air duct module. Herein, “the main plane of the air duct cover” may represent a plane where a region of the air duct cover with the largest area is located.

According to an optional embodiment of this application, the air duct main body can be provided with mounting holes. The mounting holes can completely penetrate the air duct main body in a direction perpendicular to the air duct cover.

For example, the purification module can extend into the mounting holes or pass through the mounting holes from the side of the air duct main body facing away from the air duct cover, so as to be installed to the air duct cover.

Alternatively or additionally, mounting brackets of the air duct cover extend into the mounting holes, and the purification module can be installed to the air duct cover through the mounting brackets.

According to an optional embodiment of this application, the mounting brackets can extend through and beyond the mounting holes. The purification module, without extending into first through holes, can be installed to the air duct cover by way of the mounting brackets. Thus, the first through hole can be allowed to have a smaller size. The purification module can be installed to the air duct cover in the direction perpendicular to the main plane of the air duct cover, or can be installed to the mounting brackets in a direction parallel to the air duct cover. Therefore, there are more options for an engagement way between the purification module and the mounting brackets.

According to an optional embodiment of this application, the air duct main body may include a heat insulation board at least partially located between the air duct and the air duct cover. The heat insulation board can be provided with first through holes.

Optionally, the purification module can extend into the first through holes from one side of the heat insulation board facing away from the air duct cover, so as to be installed to the air duct cover.

Alternatively or additionally, the mounting brackets of the air duct cover can extend into the first through holes, such that the purification module can be installed to the air duct cover through the mounting brackets.

According to an optional embodiment of this application, in a projection direction perpendicular to the main plane of the air duct cover, a projection of the first through hole may be smaller than that of a portion of the purification module located in the air duct. A smaller first through hole can effectively prevent the air duct cover from being condensed on one side facing the storage space at a position corresponding to the purification module.

Alternatively or additionally, at least a portion of the heat insulation board can be projected onto purification cores of the purification module in the direction perpendicular to the main plane of the air duct cover. Apparently, the at least a portion of the heat insulation board is an integral portion of the heat insulation board, rather than an independent component. Thus, at least a portion of the heat insulation board can effectively separate the air duct cover from the purification cores of the purification module in a heat insulation manner. Therefore, there is no need to additionally dispose a heat insulation component in the purification module, which can simplify the structure and significantly reduce costs. This configuration not only can prevent the air duct cover from being condensed at the side facing the storage space at the position corresponding to the purification module, but also can simplify the structure as well as the installation process.

For example, the purification cores of the purification module can be at least mostly projected onto the heat insulation board in the direction perpendicular to the main plane of the air duct cover. Thus, the heat insulation board can be effectively used for preventing the air duct cover from being condensed at the side facing the storage space at the position corresponding to the purification module.

According to an optional embodiment of this application, the heat insulation board can be provided with a receiving concave portion at a position opposite to the purification module in the direction perpendicular to the main plane of the air duct cover. The receiving concave portion is dented in a direction away from the purification module.

According to an optional embodiment of this application, the heat insulation board can be provided with protrusion columns located in the air duct. The protrusion columns penetrate the air duct in the direction perpendicular to the main plane of the air duct cover. The protrusion columns are arranged adjacent to the first through holes. Thus, the structural strength can be improved.

According to an optional embodiment of this application, the protrusion columns are arranged successively with the first through holes in a main flow direction of the air duct. Thus, impediments to flow of gas by the protrusion columns and the purification module can be reduced.

According to an optional embodiment of this application, the air duct main body may include an air duct back plate that defines the air duct at least on one side of the air duct facing away from the air duct cover. The air duct back plate can be provided with a second through hole. The purification module can pass through the second through hole or extend into the second through hole from the side of the heat insulation board facing away from the air duct cover, so as to be installed to the air duct cover.

According to an optional embodiment of this application, the purification module can seal the second through hole. Thus, the gas in the air duct can be prevented from leaking via the second through hole.

The purification module can be provided with a purification rear wall, and the purification rear wall is located on one side of the air duct back plate facing away from the air duct and completely seals the second through hole. This further helps to prevent gas leakage in the air duct.

According to an optional embodiment of this application, the refrigeration appliance may include a cover film, and the cover film is attached to the air duct back plate on the side of the air duct back plate facing away from the air duct and covers the second through hole and/or the purification module. This also helps to prevent gas leakage in the air duct.

According to an optional embodiment of this application, the purification module may include the purification cores with a purification function, and a purification bracket applicable to holding the purification cores. The purification bracket, for example, can be provided with at least one accommodating cavity applicable to accommodating the purification cores, and a quantity of the accommodating cavities is greater than or equal to that of the purification cores.

Optionally, the purification bracket can be provided with locking arms, and the locking arms lock to the mounting brackets of the air duct cover to install the purification module to the air duct cover.

According to an optional embodiment of this application, the locking arms may include a first locking arm and a second locking arm. The mounting brackets may include a first mounting bracket and a second mounting bracket engaged to the first locking arm and the second locking arm, respectively. Thus, the purification module can be reliably installed to the air duct cover by convenient operations.

According to an optional embodiment of this application, the first locking arm and the second locking arm can be located on two opposite sides of the at least one accommodating cavity, respectively. The purification module may be of a compact structure.

The first mounting bracket and the second mounting bracket, for example, may be located at outer sides of the first locking arm and the second locking arm facing away from the at least one accommodating cavity, respectively.

According to an optional embodiment of this application, the first mounting bracket and the second mounting bracket may be formed into a shape of a U groove extending in the direction perpendicular to the main plane of the air duct cover. The first locking arm and the second locking arm can be inserted into the U grooves of the first mounting bracket and the second mounting bracket, respectively. Opening directions of the U grooves of the first mounting bracket and the second mounting bracket can be arranged oppositely to each other. In the process of installing the purification module to the air duct cover or disassembling the purification module from the air duct cover, the first mounting bracket and the second mounting bracket can guide and restrict the movement of the first locking arm and the second locking arm. Thus, the purification module can be more convenient to install and disassemble. In particular, a heat insulation main body (including the heat insulation board, for example) can be prevented from being damaged in the installation and disassembly process of the purification module.

According to an optional embodiment of this application, only one of the first mounting bracket and the second mounting bracket is provided with a concave portion at an end facing the air duct, and the concave portion is open to one side facing away from the storage space. The purification bracket can be provided with a protrusion embedded into the concave portion. Thus, it can be ensured in a simple manner that the purification module is installed in a correct orientation.

According to an optional embodiment of this application, the mounting bracket can be provided with a locking opening, and the locking arm can be provided with a hook portion. The hook portion extends into the locking opening in a direction parallel to the main plane of the air duct cover. Thus, the purification module can be installed through simple steps.

According to an optional embodiment of this application, the purification bracket can be provided with a purification rear wall, and the purification rear wall is located on one side of the purification core facing away from the air duct cover. The purification rear wall, for example, can completely cover the purification cores on one side of the purification core facing away from the air duct cover.

Optionally, the locking arm can be formed into an overhang structure extending from the purification rear wall to the storage space. Thus, the purification module can be installed by way of the elastic deformation of the locking arms. This helps to reduce difficulty of operation.

The mounting brackets, for example, may abut against one side of the purification rear wall facing the purification cores. Thus, the connection between the air duct cover and the purification module may be more stable and reliable.

According to an optional embodiment of this application, a first compartment wall of the compartment may be located on one side of the air duct main body facing away from the storage space. The first compartment wall can be provided with an accommodating concave portion applicable to accommodating the purification module, and the accommodating concave portion is opposite to the purification module and is dented in the direction away from the purification module. Thus, the air duct module can be prevented from being damaged.

According to an optional embodiment of this application, the air duct module is detachably installed in the compartment. This is particularly advantageous for the repair or maintenance of the air duct module and/or the purification module.

According to an optional embodiment of this application, the purification module is detachably installed to the air duct cover. During replacement of the purification module or parts in the purification module, the purification module can be disassembled from the air duct cover, without detaching the air duct main body from the air duct cover. On the one hand, difficulty of operation can be reduced. For example, an ordinary user can replace the purification module or its parts, without the involvement of a specialized worker. On the other hand, damage to the air duct module can be also avoided in the disassembly and re-installation process of the purification module.

According to a second aspect of this application, an embodiment of this application provides a refrigeration appliance, provided with: a compartment, provided with storage space applicable to accommodating articles to be cooled; an air duct module, applicable to forming an air duct in the compartment, and including an air duct main body applicable to defining the air duct, and an air duct cover installed to one side of the air duct main body facing the storage space; and a purification module, at least partially located in the air duct to purify gas flowing through the air duct. The air duct main body includes a heat insulation board at least partially located between the air duct and the air duct cover, and at least a portion of the heat insulation board is located between the air duct cover and the purification module in a direction perpendicular to a main plane of the air duct cover.

According to a third aspect of this application, an embodiment of this application provides a refrigeration appliance, provided with: a compartment, provided with storage space applicable to accommodating articles to be cooled; an air duct module, applicable to forming an air duct in the compartment, and including an air duct main body applicable to defining the air duct, and an air duct cover installed to one side of the air duct main body facing the storage space; and a purification module, at least partially located in the air duct to purify gas flowing through the air duct. The air duct main body includes an air duct back plate that defines the air duct at least on one side of the air duct facing away from the air duct cover, and the air duct back plate is provided with a second through hole applicable to installing the purification module to the air duct cover.

According to a fourth aspect of this application, an embodiment of this application provides a method of making a refrigeration appliance, including: providing an air duct module, including an air duct main body applicable to defining an air duct, and an air duct cover installed to the air duct main body; and installing the purification module to the air duct cover from one side of the air duct main body facing away from the air duct cover in a state where the air duct main body and the air duct cover are assembled with each other, the purification module being at least partially located in the air duct.

Other features which are considered as characteristic for the invention are set forth in the appended claims.

Although the invention is illustrated and described herein as embodied in a refrigeration appliance and a method of making same, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a diagrammatic, sectional view of a refrigeration appliance according to an exemplary embodiment of the invention;

FIG. 2 is a perspective view of an air duct module and a purification module of the refrigeration appliance according to an exemplary embodiment of the present invention;

FIG. 3 is a perspective view of the air duct module and the purification module to be installed into a compartment according to an exemplary embodiment of the present invention;

FIG. 4 is a sectional view of the compartment of the refrigeration appliance according to an exemplary embodiment of the present invention;

FIG. 5 is a partially enlarged view of a circled portion shown in FIG. 4;

FIG. 6 is a perspective view of the air duct module of the refrigeration appliance according to an exemplary embodiment of the present invention;

FIG. 7 is a perspective view of the air duct module and the purification module of the refrigeration appliance according to an exemplary embodiment of the present invention;

FIG. 8 is a perspective, partially enlarged view of a framed portion shown in FIG. 7;

FIG. 9 is a rear view of the portion shown in FIG. 8;

FIG. 10 is a perspective view of the purification module of the refrigeration appliance according to an exemplary embodiment of the present invention;

FIG. 11 is sectional view of a compartment of the refrigeration appliance according to an exemplary embodiment of the present invention;

FIG. 12 is a partially enlarged view of the framed portion shown in FIG. 11;

FIG. 13 is a partially enlarged view of a circled portion shown in FIG. 12;

FIG. 14 is a perspective view of the air duct module of the refrigeration appliance according to an exemplary embodiment of the present invention;

FIG. 15 is a perspective view of the air duct module and the purification module of the refrigeration appliance according to an exemplary embodiment of the present invention;

FIG. 16 is a perspective view of the purification module installed to the air duct module according to an exemplary embodiment of the present invention;

FIG. 17 is a perspective view of a cover film attached to an air duct back plate according to an exemplary embodiment of the present invention;

FIG. 18 is a schematic illustration of another exemplary embodiment of the present invention in a view similar to FIG. 5;

FIG. 19 is a flow chart of an assembly method for the refrigeration appliance according to an exemplary embodiment of the present invention; and

FIG. 20 is a flow chart of a disassembly method for the refrigeration appliance according to an exemplary embodiment of the present invention.

### DETAILED DESCRIPTION OF THE INVENTION

To make the technical problems, technical solutions and beneficial technical effects to be addressed in this application clearer and more understandable, this application will be further described in detail below in combination with the accompanying drawings and multiple exemplary embodiments. It should be understood that the specific embodiments described here are only intended to explain this application and are not intended to limit the protection scope of this application.

First, for ease of understanding, returning to the description of the section of the background, refrigeration appliances in the prior art have the problems that the assembly process and/or disassembly process of a purification module are/is complicated, the requirements for an operator are high, and components are prone to damage in the assembly process and/or disassembly process, and so on.

For at least one of the above-mentioned technical problems and other possible technical problems, an exemplary embodiment of this application provides a refrigeration appliance, provided with: a compartment, provided with storage space applicable to accommodating articles to be cooled; an air duct module, applicable to forming an air duct in the compartment, and including an air duct main body applicable to defining the air duct, and an air duct cover installed to one side of the air duct main body facing the storage space; and a purification module, at least partially located in the air duct to purify gas flowing through the air duct. The air duct module is configured to allow the purification module to be installed to the air duct cover from one side of the air duct main body facing away from the air duct cover in a state where the air duct main body and the air duct cover are assembled with each other.

For at least one of the above-mentioned technical problems and other possible technical problems, another exemplary embodiment of this application provides a refrigeration appliance, provided with: a compartment, provided with storage space applicable to accommodating articles to be cooled; an air duct module, applicable to forming an air duct in the compartment, and including an air duct main body applicable to defining the air duct, and an air duct cover installed to one side of the air duct main body facing the storage space; and a purification module, at least partially located in the air duct to purify gas flowing through the air duct. The air duct main body includes a heat insulation board at least partially located between the air duct and the air duct cover, and at least a portion of the heat insulation board is located between the air duct cover and the purification module in a direction perpendicular to a main plane of the air duct cover.

For at least one of the above-mentioned technical problems and other possible technical problems, another exemplary embodiment of this application provides a refrigeration appliance, provided with: a compartment, provided with storage space applicable to accommodating articles to be cooled; an air duct module, applicable to forming an air duct in the compartment, and including an air duct main body applicable to defining the air duct, and an air duct cover installed to one side of the air duct main body facing the storage space; and a purification module, at least partially located in the air duct to purify gas flowing through the air duct. The air duct main body includes an air duct back plate that defines the air duct at least on one side of the air duct facing away from the air duct cover, and the air duct back plate is provided with a second through hole applicable to installing the purification module to the air duct cover.

To better understand this application, exemplary embodiments of this application will be described below with reference to the accompanying drawings.

Before starting the specific description, it should be pointed out that the directional terms used in the description process refer to the conventional use of the refrigeration appliance, are intended for the convenience of description, and cannot be understood as an absolute limitation of the corresponding characteristics. In some of the accompanying drawings, the height direction H, the width direction W, and the depth direction D perpendicular by twos are schematically illustrated in arrows.

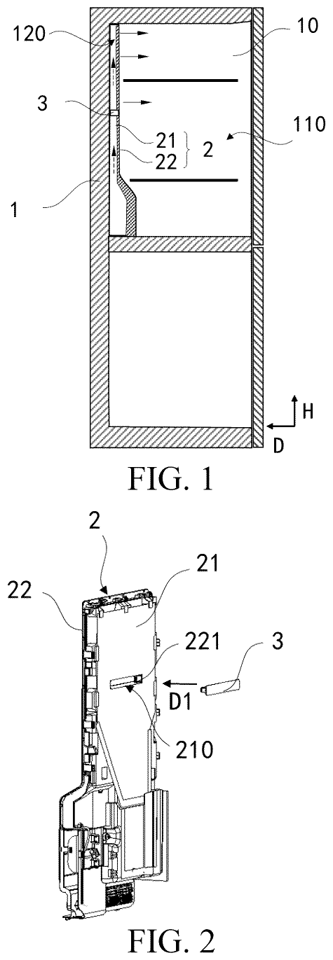

Referring now to the figures of the drawings in detail and first, particularly to FIG. 1 thereof, there is shown a refrigeration appliance according to an exemplary embodiment of this application in a sectional view.

It can be seen from FIG. 1 that the refrigeration appliance is provided with a compartment 10, provided with storage space 110 applicable to accommodating articles to be cooled. The compartment 10, for example, can be used as a cold storage chamber. The articles to be cooled can be stored in the storage space 110.

The refrigeration appliance is further provided with an air duct module 2 applicable to forming an air duct 120 in the compartment 10, and a purification module 3 at least partially located in the air duct 120 to purify gas flowing through the air duct 120. The air duct module 2 includes an air duct main body 21 applicable to defining the air duct 120, and an air duct cover 22 installed to one side of the air duct main body 21 facing the storage space 110. The air duct main body 21 defines the air duct 120 in at least one lateral direction perpendicular to a main flow direction of the air duct 120 (schematically illustrated in dotted arrows in FIG. 1), especially the air duct main body 21 defines the air duct 120 in at least three lateral directions perpendicular to the main flow direction of the air duct 120. As an example, the air duct main body 21 can define the air duct 120 in a circumferential direction perpendicular to the main flow direction of the air duct 120.

The air duct module 2 is configured to allow the purification module 3 to be installed to the air duct cover 22 from one side of the air duct main body 21 facing away from the air duct cover 22 in a state where the air duct main body 21 and the air duct cover 22 are assembled with each other. Thus, the assembly process and/or disassembly process of the air duct module 2 and the purification module 3 can be simplified.

Further explanation will be provided below in combination with FIG. 2. FIG. 2 schematically illustrates the air duct module 2 and the purification module 3 of the refrigeration appliance according to an exemplary embodiment of the present invention.

In FIG. 2, the air duct main body 21 and the air duct cover 22 are in a state where they are assembled with each other, where the air duct main body 21 and the air duct cover 22 are connected to each other in a non-relative motion manner. The purification module 3 can be installed to the air duct cover 22 from the side of the air duct main body 21 facing away from the air duct cover 22. For example, during the assembly of the refrigeration appliance, the air duct module 2 can be provided as an assembled whole, e.g., by a manufacturer. An assembly worker of the refrigeration appliance can directly operate an assembled air duct module 2, without performing assembly operations of the air duct main body 21 and the air duct cover 22. This not only can reduce difficulty of assembly operation, but also can prevent the air duct module 2 from being damaged in the assembly process.

For example, the air duct main body 21 can be provided with mounting holes 210. The purification module 3 can extend into the mounting holes 210 or pass through the mounting holes 210 from the side of the air duct main body 21 facing away from the air duct cover 22, so as to be installed to the air duct cover 22. Alternatively or additionally, the air duct cover 22 can be provided with mounting brackets 221, and the purification module 3 can be installed to the air duct cover 22 through the mounting brackets 221. The mounting brackets 221 can extend into the mounting holes 210. The mounting holes 210 can completely penetrate the air duct main body 21 particularly in a direction perpendicular to the air duct cover 22. By disposing the mounting holes 210 that completely penetrate the air duct main body 21 at a position where the purification module 3 is to be installed, the purification module 3 can be installed to a desired position with simple operation steps. Optionally, the purification module 3 can be configured to be installed to the air duct cover 22 from the side of the air duct main body 21 facing away from the air duct cover 22 in a direction D1 perpendicular to a main plane of the air duct cover 22. This helps to simplify the installation process of the purification module 3.

It is particularly advantageous that the purification module 3 is detachably installed to the air duct cover 22. During replacement of the purification module 3 or parts in the purification module 3, the purification module 3 can be directly disassembled from the air duct module 2, without separating the air duct main body 21 from the air duct cover 22.

In a case where the air duct main body 21 and the air duct cover 22 are kept in an assembled state, the disassembly and installation of the purification module 3 can be achieved. This is particularly advantageous for the repair or maintenance of the purification module 3.

On the one hand, difficulty of operation can be reduced. For example, an ordinary user can replace the purification module 3 or its parts, without the involvement of a specialized worker. On the other hand, damage to the air duct module 2 can be also avoided in the disassembly and re-installation process of the purification module 3.

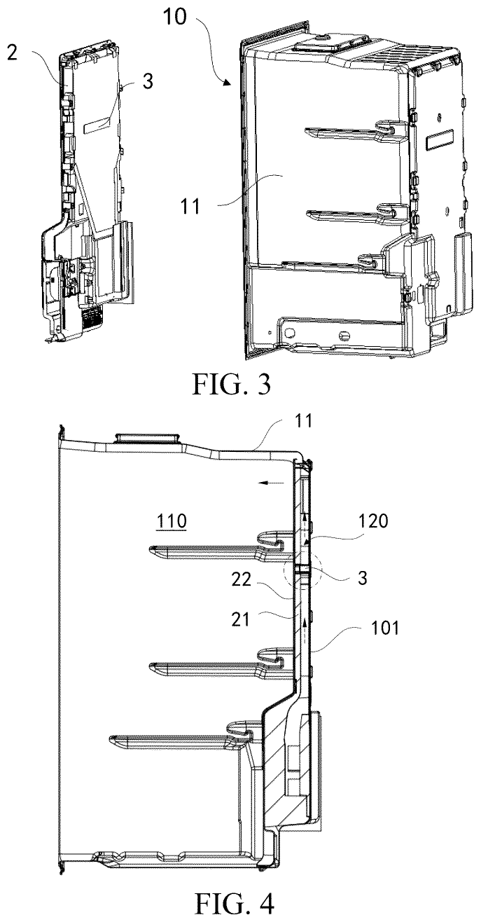

FIG. 3 schematically illustrates that the air duct module 2 and the purification module 3 are installed to a compartment 10 according to an exemplary embodiment of the present invention.

As shown in FIG. 3, the compartment 10 can be defined by an inner container 11 of the refrigeration appliance. After the purification module 3 is installed to the air duct module 2, the air duct module 2 with the purification module 3 can be installed in the compartment 10.

It is particularly advantageous that the air duct module 2 is detachably installed in the compartment 10. This is particularly advantageous for the repair or maintenance of the air duct module 2 and/or the purification module 3.

Referring to FIG. 1, the air duct module 2, for example, can be arranged at the rear portion of the compartment 10. The air duct 120 can be located between a rear wall of the compartment 10 and the air duct cover 22, and the storage space 110 is located in front of the air duct cover 22. Cooling gas, for example, can flow from the bottom to the top in the air duct 120. Herein, the air duct 120 refers in particular to a section of the air duct 120 located at a position where the purification module 3 is located.

The purification module 3 can purify gas flowing through the air duct 120. Herein, “purification” may include functions such as sterilization, disinfection, and/or deodorization. For example, the purification module 3 can produce bactericidal substances to eliminate bacteria, or be configured as a filter for filtering air. Exemplarily, the purification module 3 may include activated carbon, which reduces odors in the gas through physical adsorption. Alternatively or additionally, the purification module 3 may include an ion generation module and/or an ozone generation module, and the like. In this embodiment, the purification module 3 is arranged at the rear of the air duct cover 22, and thus, the purification module is blocked by the air duct cover 22 without being exposed to the storage space 110. The air duct cover 22 does not need to be provided with an opening in front of the purification module 3, and thus, the air duct cover may have a flat outer surface in front of the purification module (i.e., on the side facing the storage space 110).

In additional embodiments, the air duct module 2 can be also arranged at other positions of the compartment 10, such as a top portion of the compartment 10. Correspondingly, the air duct 120 can be located between a top wall of the compartment 10 and the air duct cover 22, and the storage space 110 is located below the air duct cover 22. The purification module 3 can be arranged above the air duct cover 22.

In the embodiment shown in FIG. 1, the air duct 120 can be applicable to supplying the cooling gas to the storage space 110 of the compartment 10. Optionally, the air duct 120 can be also applicable to supplying the cooling gas to additional compartments other than the compartment 10. For example, the air duct module 2 can be arranged in a freezing chamber of the refrigeration appliance, and is applicable to supplying the cooling gas to the cold storage chamber of the refrigeration appliance. Alternatively, using the air duct 120 as a return air duct is also feasible. In this case, the air duct 120 can be applicable to guiding the gas in the storage space 110 to a refrigeration device (such as an evaporator) of the refrigeration appliance.

It can be seen from FIG. 1 that the refrigeration appliance may further include a freezing chamber located on a lower side of the cold storage chamber. Those skilled in the art should understand that the above-mentioned compartment 10 can be used as a freezing chamber as well. In this embodiment, the refrigeration appliance, for example, can be implemented as a combined refrigerator with a cold storage chamber and a freezing chamber. Those skilled in the art will understand that the present invention applies equally to other types of refrigerators, such as an independent cold storage machine or a freezer. In addition, as needed, the present invention can be also applied to other refrigeration appliances other than the refrigerator, such as a wine cooler. The present invention is particularly suitable for a household refrigeration appliance.

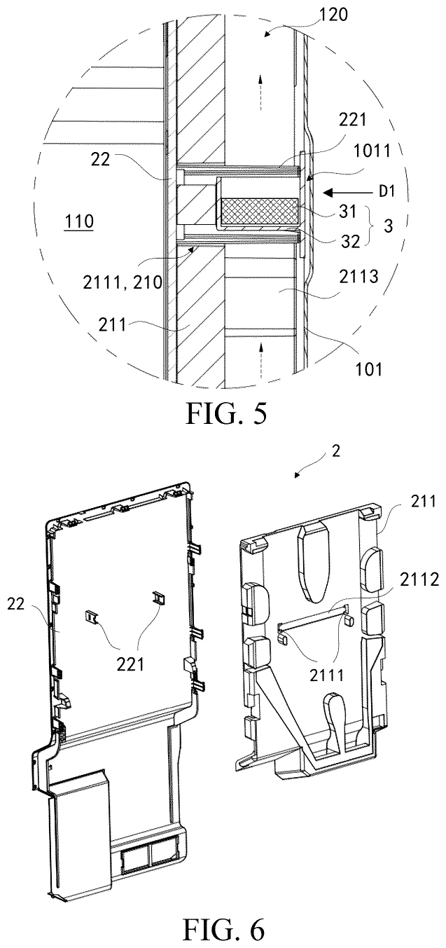

FIG. 4 schematically illustrates a compartment 10 of a refrigeration appliance according to an exemplary embodiment of the present invention in a sectional view. FIG. 5 schematically illustrates a partially enlarged view of a circled portion of FIG. 4.

As shown in FIG. 4, the compartment 10 can be defined by an inner container 11. The air duct module 2 is exemplarily arranged in front of a rear wall of the inner container 11. Therefore, the storage space 110 is located in front of the air duct cover 22, and the air duct 120 and the air duct main body 21 are located at the rear of the air duct cover 22.

In combination with FIG. 5, it can be seen that the air duct main body 21 may include a heat insulation board 211 at least partially located between the air duct 120 and the air duct cover 22. The heat insulation board 211 may be made of a heat insulation material, such as expanded polystyrene (EPS).

The heat insulation board 211 can be provided with first through holes 2111. The mounting holes 210 described above may include or be formed into the first through holes 2111.

Optionally, the purification module 3 can extend into the first through holes 2111 from one side of the heat insulation board 211 facing away from the air duct cover 22, so as to be installed to the air duct cover 22. As shown in the solid arrow in FIG. 5, the purification module 3 can be installed to the air duct cover 22 in the direction D1.

Here, the air duct cover 22 is provided with mounting brackets 221 on one side facing the air duct 120, and the purification module 3 can be installed to the air duct cover 22 by way of the mounting brackets 221. The mounting brackets 221, for example, can extend into the first through holes 2111. The air duct cover 22, for example, can be formed into a single integrated component. In other words, the mounting brackets 221 can form an integral portion of the air duct cover 22. Optionally, the air duct cover 22 can be formed into a plastic piece, e.g., an injection molded piece. Thus, the air duct cover 22 that can stably and reliably bear the purification module 3 can be formed in a convenient-to-manufacture manner.

In additional embodiments, it is also feasible that the purification module 3 is installed to the air duct cover 22 in other ways, such as magnetic attraction or threaded connection.

Referring to FIG. 5, the purification module 3 can be installed to a back side of the air duct cover 22 facing away from the storage space 110 from the side of the heat insulation board 211 facing away from the air duct cover 22, and the purification module 3 can be completely blocked by the air duct cover 22 at the front without being exposed to the storage space 110. The air duct cover 22 also does not need to be provided with an opening in front of the purification module 3, and thus, the air duct cover may have a flat outer surface in front of the purification module.

As an example, the purification module 3 may include the purification cores 31 with a purification function, and a purification bracket 32 applicable to holding the purification cores 31. The purification core 31, for example, may include a filter element, an ion generator, and/or an ozone generator. The purification bracket 32 can be engaged with the mounting brackets 221. As shown in the dotted arrow in FIG. 5, cooling gas can flow from the bottom to the top in the air duct 120. The cooling gas flowing through the purification module 3 can be purified.

In additional embodiments, it is also feasible that the purification module 3 has other configurations.

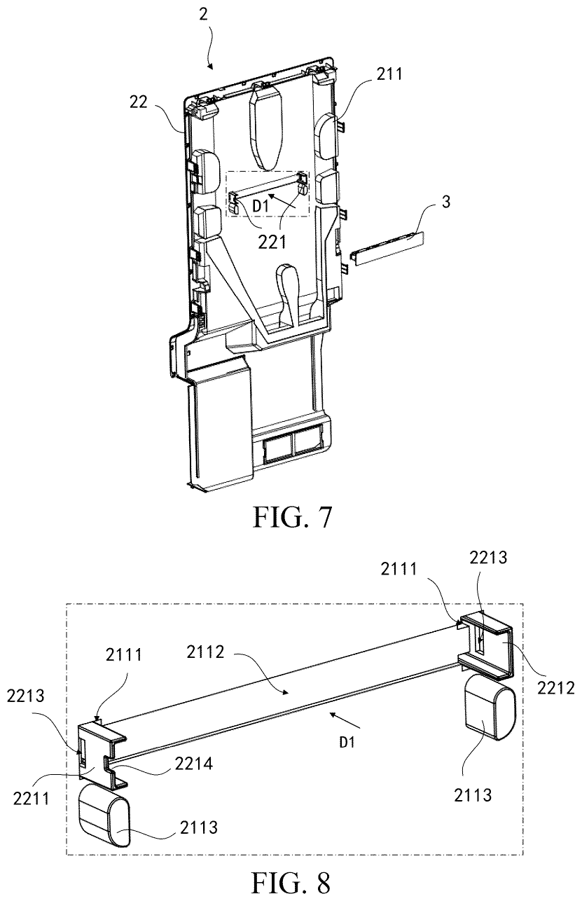

FIG. 6 schematically illustrates an air duct module 2 of a refrigeration appliance according to an exemplary embodiment of the present invention. FIG. 7 schematically illustrates an air duct module 2 and a purification module 3 of a refrigeration appliance according to an exemplary embodiment of the present invention.

The air duct module 2 is shown in an exploded view in FIG. 6, where the air duct main body 21 may include a heat insulation board 211, which has not yet been assembled to the air duct cover 22.

The heat insulation board 211, for example, can be provided with a channel, applicable to forming the air duct 120, on the side facing away from the air duct cover 22.

The air duct cover 22 can be provided with mounting brackets 221 on one side facing the air duct 120. At a position opposite to the mounting brackets 221 in the direction perpendicular to the main plane of the air duct cover 22, the heat insulation board 211 can be provided with first through holes 2111. In the embodiment shown in FIG. 6, the air duct cover 22 is provided with a pair of mounting brackets 221. The heat insulation board 211 is optionally provided with a pair of first through holes 2111 corresponding to the mounting brackets 221.

The air duct module 2 is shown in an assembled state in FIG. 7. The heat insulation board 211 can be directly connected to the air duct cover 22. In combination with FIG. 6 and FIG. 7, it can be seen that the mounting brackets 221 can extend into the first through holes 2111. The mounting brackets 221 can particularly extend through and beyond the first through holes 2111 (here, the mounting holes 210). In this case, the purification module 3 can be installed to the air duct cover 22 in a direction D1 perpendicular to a main plane of the air duct cover 22. Alternatively, the purification module 3 can be also installed to the mounting brackets 221 in a direction parallel to the air duct cover 22. In a case where the mounting brackets 221 pass through and beyond the first through holes 2111, the purification module 3, without extending into first through holes 2111, can be installed to the air duct cover 22 by way of the mounting brackets 221. Thus, the first through hole 2111 can be allowed to have a smaller size. In addition, there may be more options for the installation direction of the purification module 3.

In additional embodiments, the mounting brackets 221, for example, may terminate in the first through holes 2111 in a direction facing away from the storage space 110 (i.e., in a backward direction). That is, the mounting brackets 221 do not extend from the first through holes 2111.

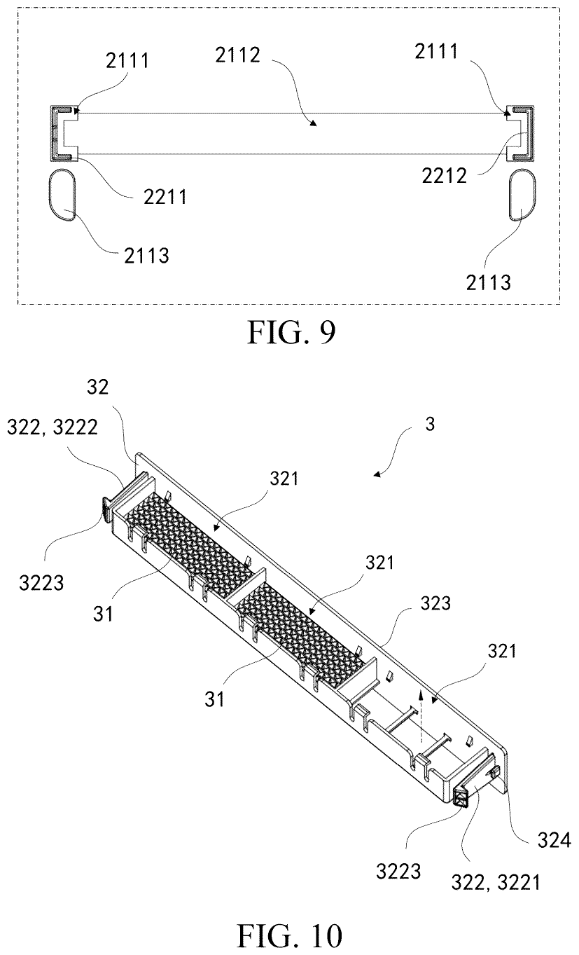

FIG. 8 schematically illustrates a partially enlarged view of a framed portion of FIG. 7. FIG. 9 schematically illustrates a rear view of a portion shown in FIG. 8.

It can be seen more clearly from FIG. 8 that the mounting brackets 221 include a first mounting bracket 2211 and a second mounting bracket 2212, and the first mounting bracket and the second mounting bracket extend through the two first through holes 2111 of the heat insulation board 211, respectively.

In combination with FIG. 7 and FIG. 8, it can be seen that in a projection direction perpendicular to the main plane of the air duct cover 22, a projection of the first through hole 2111 may be smaller than that of a portion of the purification module 3 located in the air duct 120.

In other words, when viewed in the direction perpendicular to the main plane of the air duct cover 22, the first through hole 2111 has a significantly smaller size compared to the portion of the purification module 3 located in the air duct 120. A smaller first through hole 2111 can effectively prevent the air duct cover 22 from being condensed on one side facing the storage space 110 at a position corresponding to the purification module 3.

Returning to FIG. 5, it can be seen that at least a portion of the heat insulation board (211) can be projected onto purification cores 31 of the purification module 3 in the direction perpendicular to the main plane of the air duct cover 22. In other words, the at least a portion of the heat insulation board 211 is located between the air duct cover 22 and the purification module 3 in the direction perpendicular to the main plane of the air duct cover 22. Apparently, the at least a portion of the heat insulation board 211 is an integral portion of the heat insulation board 211, rather than an independent component. Thus, at least a portion of the heat insulation board 211 can effectively separate the air duct cover 22 from the purification cores 31 of the purification module 3 in a heat insulation manner. Therefore, there is no need to additionally dispose a heat insulation component in the purification module 3, which can simplify the structure and significantly reduce costs.

This configuration not only can prevent the air duct cover 22 from being condensed at the side facing the storage space 110 at the position corresponding to the purification module 3, but also can simplify the structure as well as the installation process.

The purification cores 31 of the purification module 3 can be projected onto the heat insulation board 211 particularly at least mostly in the direction perpendicular to the main plane of the air duct cover 22. Thus, the heat insulation board 211 can be effectively used for preventing the air duct cover 22 from being condensed at the side facing the storage space 110 at the position corresponding to the purification module 3.

Referring to FIG. 6 and FIG. 8, the heat insulation board 211, for example, can be provided with a receiving concave portion 2112 at a position opposite to the purification module 3 in the direction perpendicular to the main plane of the air duct cover 22. The receiving concave portion 2112 is dented in a direction away from the purification module 3 (here, dented forwards). A bottom surface of the receiving concave portion 2112 is opposite to the purification module 3.

As shown in FIG. 9, the receiving concave portion 2112 can be located between paired first through holes 2111. The paired first through holes 2111 can define boundaries of the receiving concave portion 2112 on two opposite sides.

In an exemplary embodiment of the present invention, the heat insulation board 211 can be provided with protrusion columns 2113 located in the air duct 120, as shown in FIG. 5. The protrusion columns 2113 can penetrate the air duct 120 in the direction perpendicular to the main plane of the air duct cover 22. Optionally, the protrusion columns 2113 can be supported on the inner container 11. As shown in FIG. 8 and FIG. 9, the protrusion columns 2113 can be arranged adjacent to the first through holes 2111. Thus, the structural strength can be improved.

The protrusion columns 2113 can be arranged successively with the first through holes 2111 particularly in a main flow direction of the air duct 120.

Thus, impediments to flow of gas by the protrusion columns 2113 and the purification module 3 can be reduced. The successive arrangement of the protrusion columns 2113 with the first through holes 2111 may include a case where the protrusion columns 2113 are on the upper stream of the first through holes 2111 and a case where the protrusion columns 2113 are on the lower stream of the first through holes 2111. For example, the two protrusion columns 2113 can be disposed directly below the two first through holes 2111, respectively. The protrusion columns 2113 and the purification module 3 have a less impediment to the cooling gas flowing from bottom to top.

FIG. 10 schematically illustrates a purification module 3 of a refrigeration appliance according to an exemplary embodiment of the present invention.

As shown in FIG. 10, the purification module 3 may include at least one purification core 31 and a purification bracket 32.

The purification bracket 32 can be provided with at least one accommodating cavity 321 applicable to accommodating the purification cores 31. A quantity of the accommodating cavities 321 may be greater than or equal to that of the purification cores 31. For example, the purification bracket 32 can be provided with three accommodating cavities 321. The purification cores 31 can be arranged in two accommodating cavities 321 of the three accommodating cavities 321 as needed. The remaining one accommodating cavity 321 may be empty. Thus, an appropriate quantity of purification cores 31 can be installed as needed.

The accommodating cavities 321 can be configured to be connected in the main flow direction of the air duct 120 (as indicated by the dotted arrow in FIG. 10), such that the cooling gas in the air duct 120 can be fully contacted with the purification cores 31.

Optionally, the purification bracket 32 can be provided with locking arms 322. The locking arms 322 are applicable to locking to the mounting brackets 221 of the air duct cover 22 to install the purification module 3 to the air duct cover 22.

The locking arms 322, for example, may include a first locking arm 3221 and a second locking arm 3222. The first locking arm 3221 and the second locking arm 3222 can be engaged to the first mounting bracket 2211 and the second mounting bracket 2212, respectively. Thus, the air duct cover 22 can be reliably installed with convenient operation.

The first locking arm 3221 and the second locking arm 3222 can be particularly located on two opposite sides of the at least one accommodating cavity 321, respectively. The purification module 3 may be of a compact structure, which can reduce the occupied space of the air duct 120 and the resistance to the gas in the air duct 120.

The purification bracket 32, for example, can be provided with a purification rear wall 323, and the purification rear wall 323 is located on one side of the purification core 31 facing away from the air duct cover 22. Optionally, the purification rear wall 323 can completely cover the purification cores 31 on one side of the purification core 31 facing away from the air duct cover 22.

The locking arm 322 can be particularly formed into an overhang structure extending from the purification rear wall 323 to the storage space 110. Thus, the purification module 3 can be installed by way of the elastic deformation of the locking arms 322.

As shown in FIG. 8 and FIG. 9, the first mounting bracket 2211 and the second mounting bracket 2212 may be formed into a shape of a U groove extending in the direction perpendicular to the main plane of the air duct cover 22. In other words, in a cross section parallel to the air duct cover 22, the first mounting bracket 2211 and the second mounting bracket 2212 have a U-shaped cross section. The first locking arm 3221 and the second locking arm 3222 can be inserted into the U grooves of the first mounting bracket 2211 and the second mounting bracket 2212, respectively. Opening directions of the U grooves of the first mounting bracket 2211 and the second mounting bracket 2212 are arranged oppositely to each other. In the process of installing the purification module 3 to the air duct cover 22 or disassembling the purification module from the air duct cover 22, the first mounting bracket 2211 and the second mounting bracket 2212 can guide and restrict the movement of the first locking arm 3221 and the second locking arm 3222. Thus, the purification module 3 can be more convenient to install and disassemble. In particular, the heat insulation board 211 can be prevented from being damaged in the installation and disassembly process of the purification module 3.

According to an optional embodiment of the present invention, the mounting bracket 221 can be provided with a locking opening 2213, and the locking arm 322 can be provided with a hook portion 3223, as shown in FIG. 8 and FIG. 10. The hook portion 3223 can extend into the locking opening 2213 in a direction parallel to the main plane of the air duct cover 22. Thus, the purification module 3 can be installed through simple steps.

FIG. 8 further illustrates that only one of the first mounting bracket 2211 and the second mounting bracket 2212 is provided with a concave portion 2214 at an end facing the air duct 120, and the concave portion 2214 is open to one side facing away from the storage space 110. For example, only the first mounting bracket 2211 is provided with the concave portion 2214, while the second mounting bracket 2212 is not provided with the concave portion 2214 at a corresponding position. As shown in FIG. 10, the purification bracket 32 can be provided with a protrusion 324 embedded into the concave portion 2214. Thus, it can be ensured in a simple manner that the purification module 3 is installed in a correct orientation. If an operator attempts to install the upside-down purification module 3 to the air duct cover 22, the protrusion 324 of the purification bracket 32 will interfere with the second mounting bracket 2212 not provided with the concave portion 2214, thereby preventing the upside-down purification module 3 from being installed to the air duct cover 22.

Therefore, even if the operator finds it difficult to visually observe the mounting brackets 221 and locking arms 322 in the installation process, it can be also ensured that the purification bracket 32 is installed correctly.

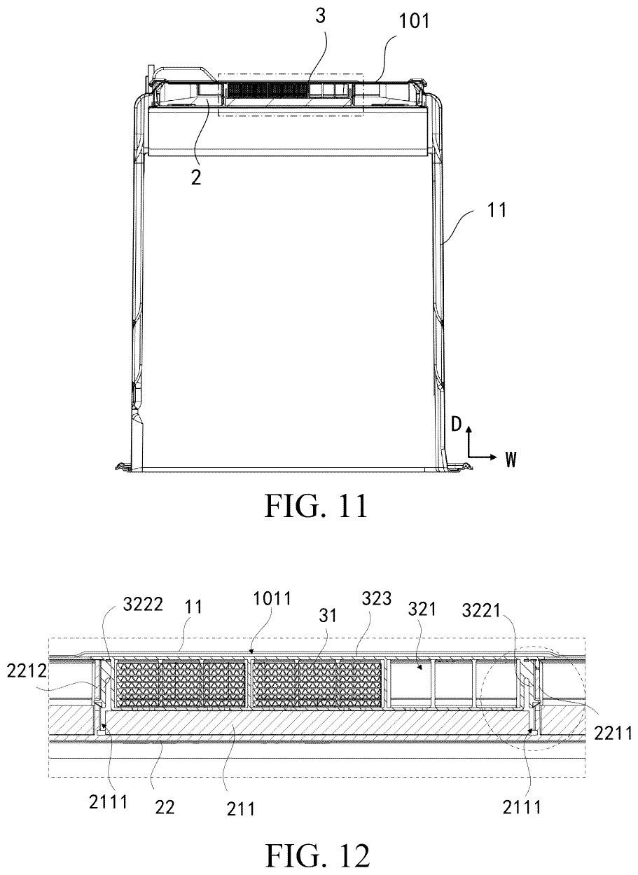

FIG. 11 schematically illustrates a compartment 10 of a refrigeration appliance according to an exemplary embodiment of the present invention in a sectional view. FIG. 12 schematically illustrates a partially enlarged view of a framed portion of FIG. 11.

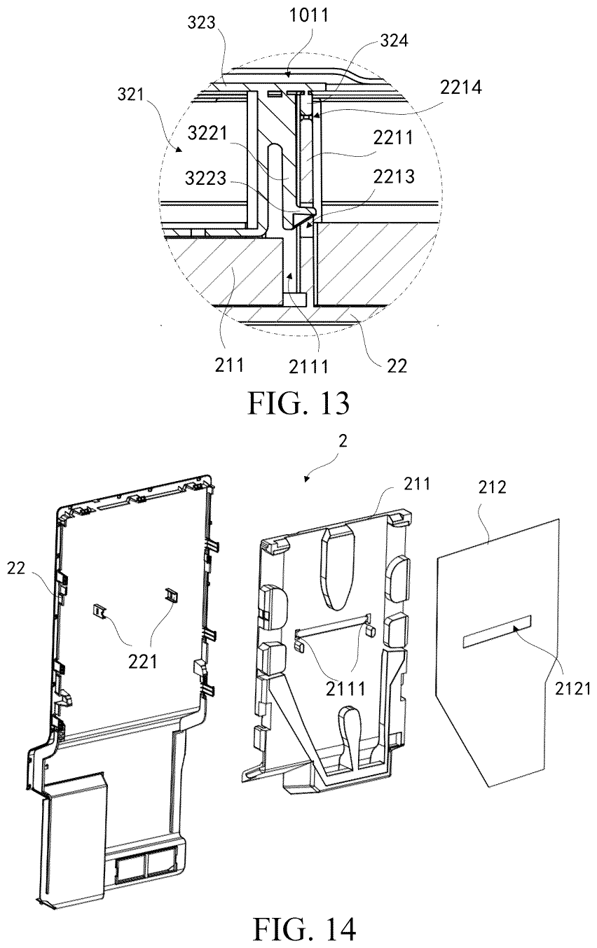

FIG. 13 schematically illustrates a partially enlarged view of a circled portion of FIG. 12.

FIG. 11 illustrates that the air duct module 2 and the purification module 3 are arranged in the inner container 11, for example, arranged at a first compartment wall 101.

It can be seen more clearly from FIG. 12 and FIG. 13 that the first mounting bracket 2211 and the second mounting bracket 2212 extend through and beyond the two first through holes 2111, respectively. The first locking arm 3221 and the second locking arm 3222 are engaged to the first mounting bracket 2211 and the second mounting bracket 2212, respectively. The first locking arm 3221 and the second locking arm 3222 do not need to extend into the first through holes 2111.

The first mounting bracket 2211 and/or the second mounting bracket 2212 may abut against one side of the purification rear wall 323 facing the purification cores 31. Thus, the connection between the air duct cover 22 and the purification module 3 may be more stable and reliable.

The first mounting bracket 2211 and the second mounting bracket 2212, for example, can be located at outer sides of the first locking arm 3221 and the second locking arm 3222 facing away from the at least one accommodating cavity 321, respectively. The hook portions 3223 of the first locking arm 3221 and the second locking arm 3222 can protrude in opposite directions. The hook portion 3223 can extend into the locking opening 2213 in a direction parallel to the main plane of the air duct cover 22. The purification module 3 can be limited at least in the direction perpendicular to the main plane of the air duct cover 22 through the hook portions 3223.

Of the first mounting bracket 2211 and the second mounting bracket 2212, only the first mounting bracket 2211 is provided with a concave portion 2214 at an end facing the air duct 120, and the concave portion 2214 is open to one side facing away from the storage space 110. The purification bracket 32 can be provided with a protrusion 324 embedded in the concave portion 2214 next to the first locking arm 3221.

According to an exemplary embodiment of the present invention, a first compartment wall 101 of the compartment 10 may be located on one side of the air duct main body 21 facing away from the storage space 110. The first compartment wall 101 can be provided with an accommodating concave portion 1011 applicable to accommodating the purification module 3, as shown in FIG. 5, FIG. 12, and FIG. 13. The accommodating concave portion 1011 may be opposite to the purification module 3 and is dented in a direction away from the purification module 3 (here, dented backwards).

Thus, the air duct module 2 can be prevented from being damaged.

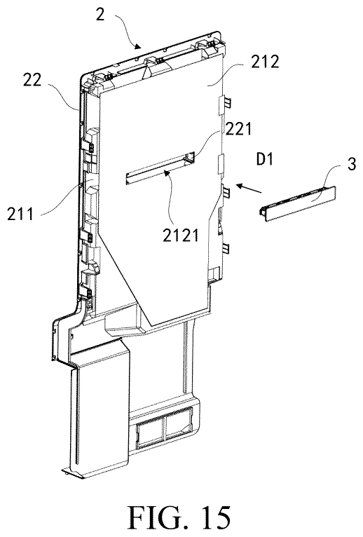

FIG. 14 schematically illustrates an air duct module 2 of a refrigeration appliance according to an exemplary embodiment of the present invention. FIG. 15 schematically illustrates an air duct module 2 and a purification module 3 of a refrigeration appliance according to an exemplary embodiment of the present invention.

The air duct module 2 is shown in an exploded view in FIG. 14. In this embodiment, the air duct main body 21 of the air duct module 2 may include a heat insulation board 211 and an air duct back plate 212. The air duct back plate 212 defines the air duct 120 at least on one side of the air duct 120 facing away from the air duct cover 22. The air duct main body 21, for example, can define the air duct 120 at least in two opposite directions perpendicular to the main plane of the air duct cover plate through the heat insulation board 211 and the air duct back plate 212. The air duct back plate 212 can be made of a heat insulation material. The air duct back plate 212 can be also made of other materials, such as plastics.

As shown in FIG. 14, the air duct back plate 212 can be provided with a second through hole 2121. In combination with FIG. 15, it can be seen that the purification module 3 can pass through the second through hole 2121 or extend into the second through hole 2121 from the side of the heat insulation board 211 facing away from the air duct cover 22, so as to be installed to the air duct cover 22. The second through hole 2121 can be disposed at a position corresponding to the purification module 3 in the direction perpendicular to the main plane of the air duct cover 22.

In this embodiment, the air duct cover 22 and the heat insulation board 211 can be constructed in the same or similar manner as described above. For example, the air duct cover 22 can be provided with mounting brackets 221, and the purification module 3 can be installed to the air duct cover 22 through the mounting brackets 221. The heat insulation board 211 can be provided with first through holes 2111, and the purification module 3 and/or the mounting brackets 221 may extend into the first through holes 2111. The mounting holes 210 of the air duct main body 21 may include the first through holes 2111 and the second through hole 2121. The first through holes 2111 can be configured to be aligned with the second through hole 2121.

The air duct back plate 212 and the heat insulation board 211 may also have other variations. For example, the heat insulation board 211 cannot be provided with a channel, but rather be formed into a substantially flat board shape. The air duct back plate 212 can be provided with a channel, applicable to forming the air duct 120, on the side facing the air duct 120.

In additional embodiments, the air duct main body 21 may not include a heat insulation board 211 located between the air duct 120 and the air duct cover 22. The air duct 120 can be defined by the air duct cover 22 in a direction facing the storage space 110, and defined by the air duct back plate 212 on one side opposite to the air duct cover 22.

The air duct module 2 is shown in an assembled state in FIG. 15. The air duct cover 22 and the air duct main body 21 (here, including the heat insulation board 211 and the air duct back plate 212) are in a state of being assembled with each other, where the air duct cover 22, the heat insulation board 211, and the air duct back plate 212 are connected to each other in a non-relative motion manner. The purification module 3 can be installed to the air duct cover 22 from the side of the air duct main body 21 facing away from the air duct cover 22.

The purification module 3 can be engaged to the mounting brackets 221 through the second through hole 2121 in a direction D1 perpendicular to the main plane of the air duct cover 22, and thus, the module is installed to the air duct cover 22.

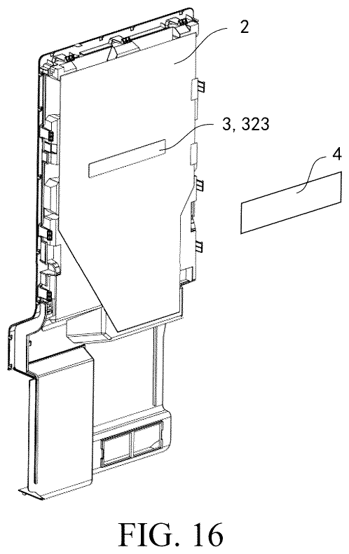

FIG. 16 schematically illustrates that a purification module 3 is installed to an air duct module 2 according to an exemplary embodiment of the present invention. In this state, the purification module 3 can be embedded into the air duct module 2, and only the purification rear wall 323 is exposed.

As shown in FIG. 16, the refrigeration appliance, for example, may include a cover film 4. The cover film 4 can be attached to the air duct back plate 212 on one side of the air duct back plate 212 facing away from the air duct 120 and covers the second through hole 2121 and/or the purification module 3.

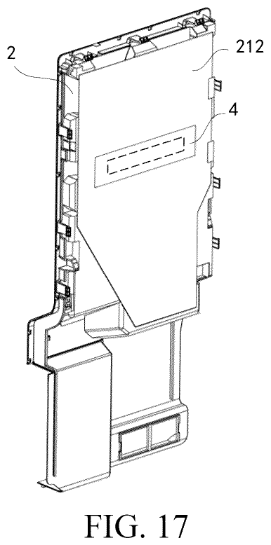

FIG. 17 schematically illustrates that a cover film 4 is attached to an air duct back plate 212 according to an exemplary embodiment of the present invention.

As shown in FIG. 17, the cover film 4 can seal any possible gaps between the purification module 3 and the air duct back plate 212 from the rear. The cover film 4 can completely cover the purification rear wall 323 particularly (schematically illustrated in dotted lines in FIG. 17). The coverage range of the cover film 4 may be slightly larger than that of the purification rear wall 323. The area of the cover film 4 is particularly not greater than 4 times the area of the purification rear wall 323. For example, the cover film 4 can be adhered to the air duct back plate 212.

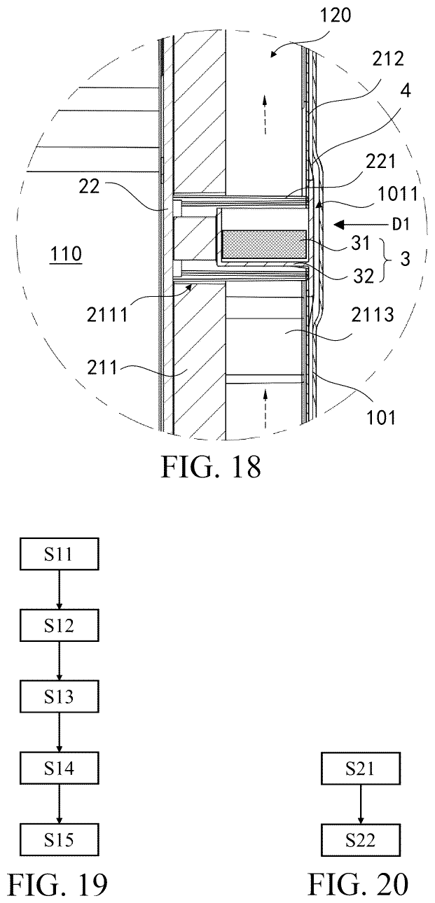

FIG. 18 schematically illustrates another exemplary embodiment of the present invention in a view similar to FIG. 5.

In this embodiment, the refrigeration appliance may have a structure similar to that of the refrigeration appliance in the embodiment shown in FIG. 5. The difference is at least that, in the refrigeration appliance shown in FIG. 18, the air duct back plate 212 and the cover film 4 are also disposed.

As shown in FIG. 18, the purification module 3 can be installed to the air duct cover 22 through the second through hole 2121 in a direction D1 perpendicular to the main plane of the air duct cover 22. The purification module 3 can particularly seal the second through hole 2121. Thus, the gas in the air duct 120 can be prevented from leaking via the second through hole 2121.

The purification module 3, for example, can be provided with a purification rear wall 323. The purification rear wall 323 can be located on the side of the air duct back plate 212 facing away from the air duct 120 and completely seals the second through hole 2121. The purification rear wall 323 can abut against the air duct back plate 212 on the side of the air duct back plate 212 facing away from the air duct 120. This further helps to prevent gas leakage in the air duct 120.

Optionally, the cover film 4 can be attached to the air duct back plate 212 on the side of the air duct back plate 212 facing away from the air duct 120 and covers the purification module 3. Any possible gaps between the purification module 3 and the air duct back plate 212 can be sealed by the cover film 4.

FIG. 18 further illustrates that a first compartment wall 101 of the compartment 10 is located on one side of the air duct main body 21 facing away from the storage space 110, and is provided with an accommodating concave portion 1011 applicable to accommodating the purification module 3. The accommodating concave portion 1011 can be dented backwards. The purification module 3 is accommodated by the accommodating concave portion 1011, which can prevent the purification module 3 from pressing the air duct main body 21, especially the air duct back plate 212. By way of the accommodating concave portion 1011, the purification module 3 will not abut against the first compartment wall 101. Thus, the air duct module 2 can be prevented from being damaged. For example, in a case where the air duct module 2 is pushed towards the first compartment wall 101, the air duct back plate 212 can be prevented from being damaged due to pressure by the purification rear wall 323.

FIG. 18 further illustrates that the heat insulation board 211 can be provided with protrusion columns 2113 located in the air duct 120. The protrusion columns 2113 can penetrate the air duct 120 in the direction perpendicular to the main plane of the air duct cover 22. The protrusion columns 2113, for example, can be supported on the air duct back plate 212. The protrusion columns 2113 can be arranged particularly adjacent to the first through holes 2111.

During the installation and disassembly of the purification module 3, the protrusion columns 2113 can prevent the air duct module 2 from being damaged.

FIG. 19 schematically illustrates an assembly method for a refrigeration appliance according to an exemplary embodiment of the present invention.

According to an embodiment of the present invention, after the air duct main body 21 and the air duct cover are assembled with each other into the air duct module 2, the purification module 3 is installed from the side of the air duct main body 21 to the air duct module 2.

As shown in FIG. 19, the assembly method may include the following steps S11 to S15.

In step S11, a compartment 10 is provided. Step S11 may include providing a case body 1 provided with the compartment 10.

In step S12, an air duct module 2 is provided, and the air duct module 2 includes an air duct main body 21 and an air duct cover 22 assembled with each other. The air duct module 2 can be provided as an assembled whole, e.g., by a manufacturer of the air duct module 2. An assembly worker of the refrigeration appliance can directly operate an assembled air duct module 2, without performing assembly operations of the air duct main body 21 and the air duct cover 22.

In step S13, a purification module 3 is provided. The purification module 3 and the air duct module 2, for example, can be provided as independent assemblies by different manufacturers, respectively.

In step S14, the purification module 3 is installed to the air duct module 2 in a state where the air duct main body 21 and the air duct cover 22 are assembled with each other.

In step S15, the air duct module 2 with the purification module 3 is installed in the compartment 10.

FIG. 20 schematically illustrates a disassembly method for a refrigeration appliance according to an exemplary embodiment of the present invention.

As shown in FIG. 20, the disassembly method may include the following steps S21 and S22.

In step S21, the air duct module 2 with the purification module 3 is disassembled from the case body 1.

In step S22, the purification module 3 is disassembled from the air duct module 2 in a state where the air duct main body 21 and the air duct cover 22 are assembled with each other. Thus, the purification module 3 can be disassembled. Further, maintenance or repair operations can be performed on the purification module 3. For example, purification cores 31 of the purification module 3 can be replaced.

The disassembly process of the purification module 3 will not involve the separation operation of the air duct main body 21 and the air duct cover 22. Thus, difficulty of operation can be reduced. For example, an ordinary user can replace the purification core 31 of the purification module 3, without the involvement of a specialized worker. On the other hand, damage to the air duct module 2 can be also avoided in the disassembly and re-installation process of the purification module 3.

Herein, the features and advantages described for the refrigeration appliance apply equally to the assembly method and disassembly method for the refrigeration appliance, and vice versa.

Although specific embodiments have been described above, these embodiments are not intended to limit the scope of the disclosure of this application, even if only a single embodiment is described with respect to specific features. The feature examples provided in the disclosure of this application are intended to be illustrative, not limiting, unless otherwise stated. In specific implementations, multiple features can be combined with each other according to actual needs and where technically feasible. In particular, features in different embodiments may be also combined with each other. Various substitutions, alterations, and modifications can be also conceived without departing from the spirit and scope of this application.

The following is a summary list of reference numerals and the corresponding structure used in the above description of the invention:
1. Case body
10. Compartment
110. Storage space
120. Air duct
101. First compartment wall
1011. Accommodating concave portion
11. Inner container
2. Air duct module
21. Air duct main body
210. Mounting hole
211. Heat insulation board
2111. First through hole
2112. Receiving concave portion
2113. Protrusion column
212. Air duct back plate
2121. Second through hole
22. Air duct cover
221. Mounting bracket
2211. First mounting bracket
2212. Second mounting bracket
2213. Locking opening
2214. Concave portion
3. Purification module
31. Purification core
32. Purification bracket
321. Accommodating cavity
322. Locking arm
3221. First locking arm
3222. Second locking arm
3223. Hook portion
323. Purification rear wall
324. Protrusion
4. Cover film

1. A refrigeration appliance, comprising:
a compartment defining a storage space applicable to accommodating articles to be cooled;
an air duct module forming an air duct in said compartment, and containing an air duct main body defining said air duct, and an air duct cover installed to one side of said air duct main body facing the storage space; and
a purification module, at least partially disposed in said air duct to purify gas flowing through said air duct, wherein said air duct module is configured to allow said purification module to be installed to said air duct cover from one side of said air duct main body facing away from said air duct cover in a state where said air duct main body and said air duct cover are assembled with each other.

2. The refrigeration appliance according to claim 1, wherein said purification module is configured installed to said air duct cover from said side of said air duct main body facing away from said air duct cover in a direction perpendicular to a main plane of said air duct cover.

3. The refrigeration appliance according to claim 1, wherein said air duct main body has mounting holes formed therein, such that said purification module is configured to extend into said mounting holes or pass through said mounting holes from said side of said air duct main body facing away from said air duct cover, so as to be installed to said air duct cover.

4. The refrigeration appliance according to claim 1, wherein:
said air duct main body has mounting holes formed therein;
said air duct cover has mounting brackets extending into said mounting holes; and
said purification module is configured to be installed to said air duct cover through said mounting brackets.

5. The refrigeration appliance according to claim 4, wherein said mounting brackets extend through and beyond said mounting holes.

6. The refrigeration appliance according to claim 1, wherein:
said air duct main body contains a heat insulation board at least partially disposed between said air duct and said air duct cover, said heat insulation board has first through holes formed therein;
said purification module is configured to extend into said first through holes from one side of said heat insulation board facing away from said air duct cover, so as to be installed to said air duct cover; and/or
said air duct cover has mounting brackets which extend into said first through holes, such that said purification module is configured to be installed to said air duct cover through said mounting brackets.

7. The refrigeration appliance according to claim 6, wherein:
in a projection direction perpendicular to a main plane of said air duct cover, a projection of at least one of said first through holes is smaller than that of a portion of said purification module disposed in said air duct; and/or
said purification module has purification cores, at least a portion of said heat insulation board is projected onto said purification cores of said purification module in the projection direction perpendicular to the main plane of said air duct cover; and/or
said purification cores of said purification module are at least mostly projected onto said heat insulation board in the projection direction perpendicular to the main plane of said air duct cover.

8. The refrigeration appliance according to claim 6, wherein said heat insulation board has a receiving concave portion at a position opposite to said purification module in a direction perpendicular to a main plane of said air duct cover, and said receiving concave portion is dented in a direction away from said purification module.

9. The refrigeration appliance according to claim 6, wherein said heat insulation board has protrusion columns disposed in said air duct, said protrusion columns penetrate said air duct in a direction perpendicular to a main plane of said air duct cover, and said protrusion columns are disposed adjacent to said first through holes.

10. The refrigeration appliance according to claim 9, wherein said protrusion columns are disposed successively with said first through holes in a main flow direction of said air duct.

11. The refrigeration appliance according to claim 1, wherein:
said air duct main body has an air duct back plate that defines said air duct at least on one side of said air duct facing away from said air duct cover, said air duct back plate having a second through hole formed therein; and
said purification module is configured to pass through said second through hole or extend into said second through hole from said side of said heat insulation board facing away from said air duct cover, so as to be installed to said air duct cover.

12. The refrigeration appliance according to claim 11, wherein:
said purification module seals said second through hole; and/or
said purification module has a purification rear wall, said purification rear wall is disposed on said one side of said air duct back plate facing away from said air duct and completely seals said second through hole.

13. The refrigeration appliance according to claim 11, further comprising a cover film attached to said air duct back plate on said side of said air duct back plate facing away from said air duct and covers said second through hole and/or said purification module.

14. The refrigeration appliance according to claim 1, wherein:
said air duct cover has mounting brackets; and
said purification module contains said purification cores with a purification function, and a purification bracket applicable to holding said purification cores, wherein said purification bracket has:
at least one accommodating cavity formed therein, applicable to accommodating said purification cores, a quantity of said at least one accommodating cavity being greater than or equal to that of said purification cores; and/or
locking arms for locking to said mounting brackets of said air duct cover to install said purification module to said air duct cover.

15. The refrigeration appliance according to claim 14, wherein said locking arms have a first locking arm and a second locking arm, and said mounting brackets include a first mounting bracket and a second mounting bracket engaged to said first locking arm and said second locking arm, respectively.

16. The refrigeration appliance according to claim 15, wherein:
said first locking arm and said second locking arm are disposed on two opposite sides of said at least one accommodating cavity, respectively; and/or
said first mounting bracket and said second mounting bracket are disposed at outer sides of said first locking arm and said second locking arm facing away from said at least one accommodating cavity, respectively.

17. The refrigeration appliance according to claim 15, wherein:
said first mounting bracket and said second mounting bracket are formed into a shape of a U groove extending in a direction perpendicular to a main plane of said air duct cover; and
said first locking arm and said second locking arm are inserted into said U groove of said first mounting bracket and of said second mounting bracket, respectively, and opening directions of said U grooves of said first mounting bracket and said second mounting bracket are disposed oppositely to each other.

18. The refrigeration appliance according to claim 15, wherein:
only one of said first mounting bracket and said second mounting bracket has a concave portion at an end facing said air duct, said concave portion is open to one side facing away from said storage space; and
said purification bracket has protrusion embedded into said concave portion.

19. The refrigeration appliance according to claim 14, wherein:
said mounting bracket has a locking opening formed therein; and
said locking arm has a hook portion, said hook portion extends into said locking opening in a direction parallel to a main plane of said air duct cover.

20. The refrigeration appliance according to claim 14, wherein:
said purification bracket has a purification rear wall, said purification rear wall is disposed on one side of said purification core facing away from said air duct cover;
said locking arm is formed into an overhang structure extending from said purification rear wall to said storage space; and/or
said purification rear wall completely covers said purification cores on one side of said purification core facing away from said air duct cover; and/or
said mounting brackets abut against one side of said purification rear wall facing said purification cores.

21. The refrigeration appliance according to claim 1, wherein said compartment has a first compartment wall disposed on one side of said air duct main body facing away from said storage space, said first compartment wall has an accommodating concave portion accommodating said purification module, and said accommodating concave portion is opposite to said purification module and is dented in a direction away from said purification module.

22. The refrigeration appliance according to claim 1, wherein:
said air duct module is detachably installed in said compartment; and/or
said purification module is detachably installed to said air duct cover.

23. A refrigeration appliance, comprising:
a compartment defining a storage space for accommodating articles to be cooled;
an air duct module forming an air duct in said compartment, and containing an air duct main body applicable to defining said air duct, and an air duct cover installed to one side of said air duct main body facing said storage space; and
a purification module at least partially disposed in said air duct to purify gas flowing through said air duct, wherein said air duct main body contains a heat insulation board at least partially located between said air duct and said air duct cover, and at least a portion of said heat insulation board is disposed between said air duct cover and said purification module in a direction perpendicular to a main plane of said air duct cover.

24. A refrigeration appliance, comprising:
a compartment defining a storage space applicable to accommodating articles to be cooled;
an air duct module forming an air duct in said compartment, and containing an air duct main body applicable to defining said air duct, and an air duct cover installed to one side of said air duct main body facing said storage space; and
a purification module, at least partially located in said air duct to purify gas flowing through said air duct, wherein said air duct main body contains an air duct back plate that defines said air duct at least on one side of said air duct facing away from said air duct cover, said air duct back plate has a second through hole for installing said purification module to said air duct cover at a position corresponding to said purification module in a direction perpendicular to a main plane of said air duct cover.

25. A method of making a refrigeration appliance, which comprises the steps of:
providing an air duct module having an air duct main body defining an air duct, and an air duct cover installed to the air duct main body; and
installing a purification module to the air duct cover from one side of the air duct main body facing away from the air duct cover in a state where the air duct main body and the air duct cover are assembled with each other, the purification module being at least partially located in the air duct.
